(19) United States
(12) Patent Application Publication
(10) Pub. No.: US 2011/0218549 A1
(43) Pub. Date: Sep. 8, 2011

(54) SYSTEMS AND METHODS FOR MAKING AND USING A TRIAL STIMULATION SYSTEM HAVING AN ELECTRICAL CONNECTOR DISPOSED ON A TRIAL STIMULATION LEAD

(75) Inventor: John Michael Barker, Ventura, CA (US)

(73) Assignee: Boston Scientific Neuromodulation Corporation, Valencia, CA (US)

(21) Appl. No.: 13/040,760

(22) Filed: Mar. 4, 2011

Related U.S. Application Data
(60) Provisional application No. 61/310,835, filed on Mar. 5, 2010.

(51) Int. Cl. A61N 1/05; A61B 19/00

(52) U.S. Cl. 606/129; 607/116

(57) ABSTRACT

A trial stimulation lead assembly for providing electrical stimulation of patient tissue during a trial stimulation includes a trial stimulation lead for insertion into a patient. The trial stimulation lead includes an elongated lead body having a length and a longitudinal axis. A plurality of electrodes are disposed at a distal end of the lead body. An electrical connector is disposed at a proximal end of the lead body. The electrical connector includes an outer case and a contact array disposed along the outer case. The contact array extends transversely to the longitudinal axis of the lead body. A plurality of electrical conductors extend along the length of the lead body and couple each of the plurality of electrodes to at least one of the plurality of contacts. A lumen extends along at least a portion of the length of the lead body.

### CROSS-REFERENCE TO RELATED APPLICATIONS

This application claims the benefit under 35 U.S.C. §119(e) of U.S. Provisional Patent Application Ser. No. 61/310,835 filed on Mar. 5, 2010, which is incorporated herein by reference.

### FIELD

The present invention is directed to the area of insertable electrical stimulation systems and methods of making and using the systems. The present invention is also directed to insertable trial stimulation leads having electrical connectors that couple to external trial stimulators during operation, as well as methods of making and using the trial stimulation leads, electrical connectors, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a trial stimulation lead assembly for providing electrical stimulation of patient tissue during a trial stimulation includes a trial stimulation lead configured and arranged for insertion into a patient. The trial stimulation lead includes an elongated lead body having a length, a circumference, and a longitudinal axis defined by a proximal end and a distal end. A plurality of electrodes are disposed at the distal end of the lead body. An electrical connector is disposed at the proximal end of the lead body. The electrical connector includes an outer case and a contact array disposed along the outer case. The contact array extends transversely to the longitudinal axis of the lead body. A plurality of electrical conductors extend along the length of the lead body and couple each of the plurality of electrodes to at least one of the plurality of contacts. A lumen extends along at least a portion of the length of the lead body.

In another embodiment, a kit for providing electrical stimulation of patient tissue during a trial stimulation includes a trial stimulation lead assembly. The trial stimulation lead includes an elongated lead body having a length, a circumference, and a longitudinal axis defined by a proximal end and a distal end. A plurality of electrodes are disposed at the distal end of the lead body. An electrical connector is disposed at the proximal end of the lead body. The electrical connector includes an outer case and a contact array disposed along the outer case. The contact array extends transversely to the longitudinal axis of the lead body. A plurality of electrical conductors extend along the length of the lead body and couple each of the plurality of electrodes to at least one of the plurality of contacts. A lumen extends along at least a portion of the length of the lead body. The kit also includes a lead introducer for facilitating insertion of the trial stimulation lead into the patient. The lead introducer includes an outer member configured and arranged for insertion into the patient. The lead introducer also includes an insertion needle configured and arranged for insertion into the outer member. The insertion needle is configured and arranged to receive the distal end of the trial stimulation lead.

In yet another embodiment, a method for implanting a trial electrical stimulation into a patient includes inserting an insertion needle into an outer member. A distal end of the outer member is guided to a target stimulation region within the patient. A distal end of a trial stimulation lead is inserted into the insertion needle. The trial stimulation lead includes an elongated lead body having a length, a circumference, and a longitudinal axis defined by a proximal end and a distal end. A plurality of electrodes are disposed at the distal end of the lead body. An electrical connector is disposed at the proximal end of the lead body. The electrical connector includes an outer case and a contact array disposed along the outer case. The contact array extends transversely to the longitudinal axis of the lead body. A plurality of electrical conductors extend along the length of the lead body and couple each of the plurality of electrodes to at least one of the plurality of contacts. A lumen extends along at least a portion of the length of the lead body. The outer member is removed from the patient while leaving the trial stimulation lead within the patient such that the plurality of electrodes are at the target stimulation region. The trial stimulation lead is separated from the insertion needle. The insertion needle is removed from the trial stimulation lead while leaving the trial stimulation lead within the patient such that the plurality of electrodes are at the target stimulation region.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic view of one embodiment of a proximal portion of a lead and a control module of an electrical stimulation system, according to the invention;

FIG. 2B is a schematic view of one embodiment of a proximal portion of a lead and a lead extension of an electrical stimulation system, according to the invention;

FIG. 3A is a schematic view of one embodiment of a trial stimulation system, according to the invention;

FIG. 3B is a schematic view of another embodiment of a trial stimulation system, according to the invention;

FIG. 4 is a schematic view of one embodiment of a trial stimulation lead with an electrical connector for coupling with an external trial stimulator, according to the invention;

FIG. 5A is a schematic view of one embodiment of the electrical connector of FIG. 4, the electrical connector having contacts configured into a rectangular array and an access port for a stylet defined in the contact array, according to the invention;

FIG. 5B is a schematic view of another embodiment of the electrical connector of FIG. 4, the electrical connector having contacts configured into a rectangular array and an access port for a stylet defined along an outer surface of the electrical connector, according to the invention;

FIG. 6A is a schematic view of yet another embodiment of the electrical connector of FIG. 4, the electrical connector having contacts configured into a round array and an access port for a stylet defined in the contact array, according to the invention;

FIG. 6B is a schematic view of another embodiment of the electrical connector of FIG. 4, the electrical connector having contacts configured into a round array and an access port for a stylet defined along an outer surface of the electrical connector, according to the invention;

FIG. 7 is a schematic perspective view of one embodiment of a lead introducer that includes an outer member that splits to separate from a trial lead, according to the invention;

FIG. 8A is a schematic perspective view of one embodiment of a lead and a body element of an insertion needle, the body element defining an open channel extending along a length of the body element, the open channel configured and arranged to receive the trial lead, according to the invention;

FIG. 8B is a schematic transverse cross-sectional view of several exemplary embodiments of the open channel of the body element of FIG. 8A, according to the invention;

FIG. 9A is a schematic longitudinal cross-sectional view of one embodiment of a lead introducer with an outer member disposed over a split-release insertion needle, according to the invention;

FIG. 9B is a schematic transverse cross-sectional view of one embodiment of the lead introducer of FIG. 9A, according to the invention; and

FIG. 10 is a schematic overview of one embodiment of components of a stimulation system, including an electronic subassembly disposed within a control module, according to the invention.

### DETAILED DESCRIPTION

The present invention is directed to the area of insertable electrical stimulation systems and methods of making and using the systems. The present invention is also directed to insertable trial stimulation leads having electrical connectors that couple to external trial stimulators during operation, as well as methods of making and using the trial stimulation leads, electrical connectors, and electrical stimulation systems.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; and U.S. Patent Applications Publication Nos. 2003/0114905, 2005/0165465, 2007/0150036; 2007/0161294; 2007/0219595; 2007/0239243; 2007/0150007; and 2008/0071320, and U.S. patent application Ser. No. 11/238,240, all of which are incorporated by reference.

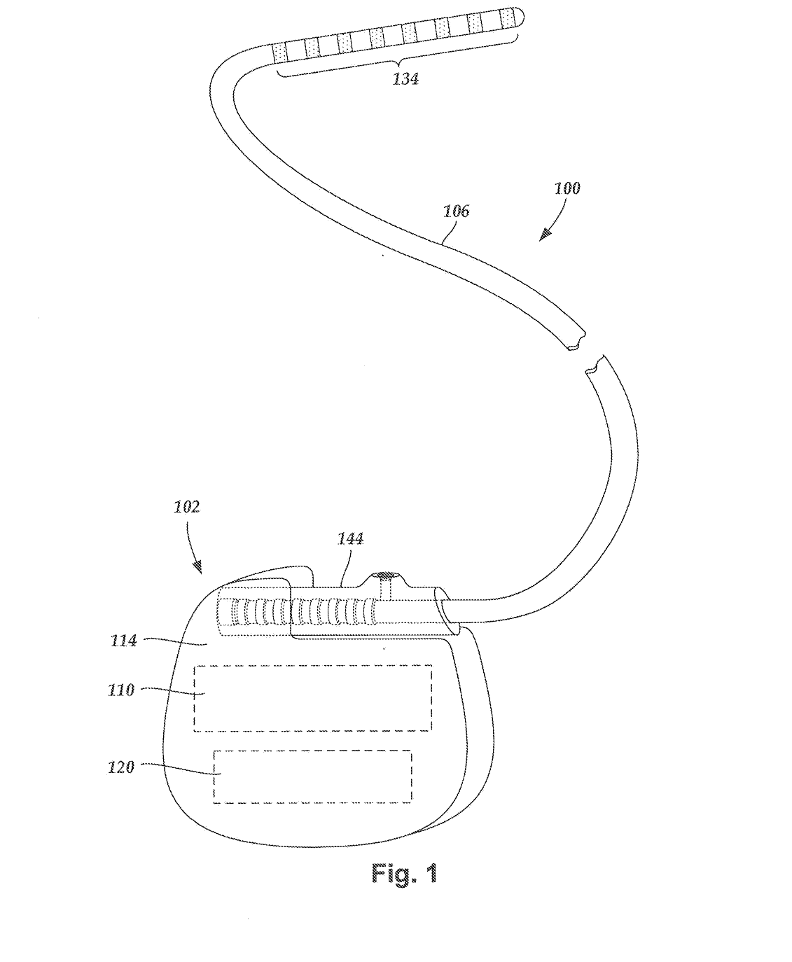

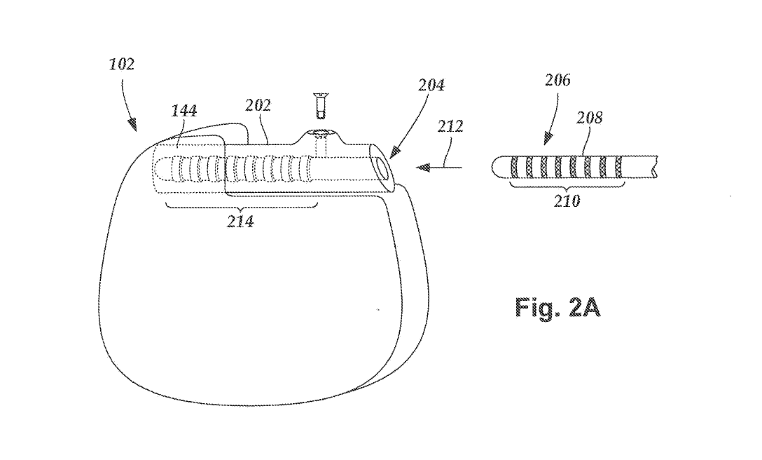

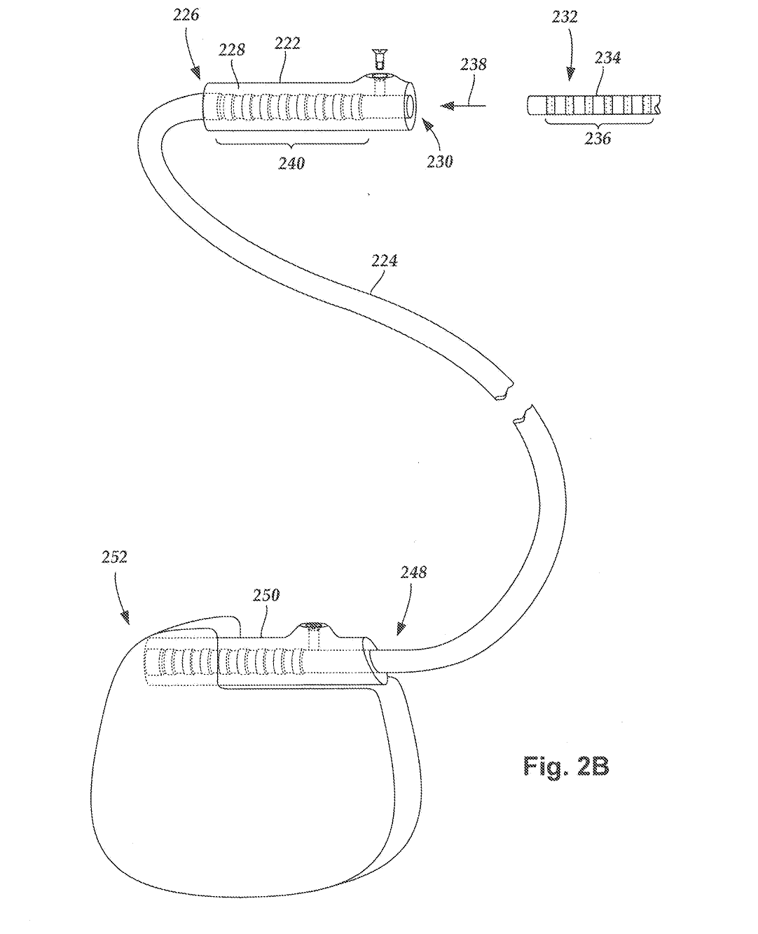

FIG. 1 illustrates schematically one embodiment of an electrical stimulation system 100. The electrical stimulation system includes a control module (e.g., a stimulator or pulse generator) 102 and at least one lead 106 coupled to the control module 102. Each lead 106 typically includes an array of electrodes 134. The control module 102 typically includes an electronic subassembly 110 and an optional power source 120 disposed in a sealed housing 114. The control module 102 typically includes a connector 144 (FIG. 2A, see also 222 and 250 of FIG. 2B) into which the proximal end of the one or more leads 106 can be plugged to make an electrical connection via conductive contacts on the control module 102 and terminals (e.g., 210 in FIGS. 2A and 236 of FIG. 2B) on each of the one or more leads 106. In at least some embodiments, a lead is isodiametric along a longitudinal length of the lead 106. In addition, one or more lead extensions 224 (see FIG. 2B) can be disposed between the one or more leads 106 and the control module 102 to extend the distance between the one or more leads 106 and the control module 102 of the embodiment shown in FIG. 1.

The electrical stimulation system or components of the electrical stimulation system, including one or more of the leads 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of one or more leads 106 are typically disposed in, or separated by, a non-conductive, biocompatible material such as, for example, silicone, polyurethane, polyetheretherketone (“PEEK”), epoxy, and the like or combinations thereof. The leads 106 may be formed in the desired shape by any process including, for example, molding (including injection molding), casting, and the like. The non-conductive material typically extends from the distal end of the one or more leads 106 to the proximal end of each of the one or more leads 106.

Terminals (e.g., 210 in FIGS. 2A and 236 of FIG. 2B) are typically disposed at the proximal end of the one or more leads 106 of the electrical stimulation system 100 for connection to corresponding conductive contacts (e.g., 214 in FIGS. 2A and 240 of FIG. 2B) in connectors (e.g., 144 in FIGS. 1-2A and 222 and 250 of FIG. 2B) disposed on, for example, the control module 102 (or to conductive contacts on a lead extension, an operating room cable, or an adaptor). Conductor wires (not shown) extend from the terminals (e.g., 210 in FIGS. 2A and 236 of FIG. 2B) to the electrodes 134. Typically, one or more electrodes 134 are electrically coupled to a terminal (e.g., 210 in FIGS. 2A and 236 of FIG. 2B). In at least some embodiments, each terminal (e.g., 210 in FIGS. 2A and 236 of FIG. 2B) is only connected to one electrode 134. The conductor wires may be embedded in the non-conductive material of the lead 106 or can be disposed in one or more lumens (not shown) extending along the lead 106. In some embodiments, there is an individual lumen for each conductor wire. In other embodiments, two or more conductor wires may extend through a lumen. There may also be one or more lumens (not shown) that open at, or near, the proximal end of the lead 106, for example, for inserting a stylet rod to facilitate placement of the lead 106 within a body of a patient. Additionally, there may also be one or more lumens (not shown) that open at, or near, the distal end of the lead 106, for example, for infusion of drugs or medication into the site of implantation of the one or more leads 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 2A, a lead 208 is shown configured and arranged for insertion to the control module 102. The connector 144 includes a connector housing 202. The connector housing 202 defines at least one port 204 into which a proximal end 206 of a lead 208 with terminals 210 can be inserted, as shown by directional arrow 212. The connector housing 202 also includes a plurality of conductive contacts 214 for each port 204. When the lead 208 is inserted into the port 204, the conductive contacts 214 can be aligned with the terminals 210 on the lead 208 to electrically couple the control module 102 to the electrodes (134 of FIG. 1) disposed at a distal end of the lead 208. Examples of connectors in control modules are found in, for example, U.S. Pat. No. 7,244,150 and U.S. patent application Ser. No. 11/532,844, which are incorporated by reference.

In FIG. 2B, a connector 222 is disposed on a lead extension 224. The connector 222 is shown disposed at a distal end 226 of the lead extension 224. The connector 222 includes a connector housing 228. The connector housing 228 defines at least one port 230 into which a proximal end 232 of a lead 234 with terminals 236 can be inserted, as shown by directional arrow 238. The connector housing 228 also includes a plurality of conductive contacts 240. When the lead 234 is inserted into the port 230, the conductive contacts 240 disposed in the connector housing 228 can be aligned with the terminals 236 on the lead 234 to electrically couple the lead extension 224 to the electrodes (134 of FIG. 1) disposed at a distal end (not shown) of the lead 234.

In at least some embodiments, the proximal end of a lead extension is similarly configured and arranged as a proximal end of a lead. The lead extension 224 may include a plurality of conductive wires (not shown) that electrically couple the conductive contacts 240 to a proximal end 248 of the lead extension 224 that is opposite to the distal end 226. In at least some embodiments, the conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a connector disposed in another lead extension. In other embodiments, the proximal end 248 of the lead extension 224 is configured and arranged for insertion into a connector disposed in a control module. As an example, in FIG. 2B the proximal end 248 of the lead extension 224 is inserted into a connector 250 disposed in a control module 252.

Sometimes trial stimulation leads are inserted into patients on a short term basis prior to implantation of the stimulation system described above with reference to FIGS. 1-2B to determine whether or not electrical stimulation is effective for treatment of one or more adverse patient conditions, such as chronic pain. Conventional trial stimulation leads are often similar to the stimulation system described above with reference to FIGS. 1-2B and include one or more electrodes (see e.g., electrodes 134 of FIG. 1) disposed at a distal end of the lead and one or more terminals (see e.g., terminals 210 of FIG. 2A) disposed at a proximal end of the lead. During a typical trial stimulation, the proximal ends of the conventional trial stimulation leads are coupled to one or more lead extensions (see e.g., lead extension 224 of FIG. 2B) which, in turn, are coupled to one or more operating room cables (“cables”) which, in turn, are coupled to an external trial stimulator.

Conventional trial stimulation leads may be inserted into a patient using an epidural needle within which the trial lead is disposed. Once the trial stimulation lead is positioned, the epidural needle may be removed from the patient by sliding the epidural needle off the proximal end of the trial stimulation lead. In at least some cases, the trial stimulation lead is isodiametric to facilitate sliding of the epidural lead over the trial stimulation lead.

After completion of a successful trial stimulation period, the trial stimulation lead can be removed and replaced with a new stimulation system (e.g., the stimulation system described above with reference to FIGS. 1-2B). Typically, the trial stimulation lead, the one or more lead extensions, and the one or more cables used during the trial stimulation are discarded after removal from the patient due to difficulty in cleaning or re-sterilization of the used equipment.

A trial stimulation system (“trial system”) includes a trial stimulation lead (“trial lead”) configured and arranged for coupling to an external trial stimulator. The trial lead includes a plurality of electrodes disposed at a distal end and an electrical connector disposed at a proximal end of the trial lead. The trial lead is long enough so that the electrical connector remains external to the patient during operation.

In at least some embodiments, the electrical connector is a conventional, commercially-available electrical connector used for electronic devices (e.g., a high definition multimedia interface (“HDMI”) connector, a LEMO connector, or the like). In at least some embodiments, the electrical connector has a number of contacts that is no fewer than the number of electrodes disposed on the trial lead. In at least some embodiments, the electrical connector has a circumference that is larger than a circumference of a body of the trial lead. In some embodiments, the electrical connector is configured and arranged to couple to an external trial stimulator via one or more cables. In at least some embodiments, the electrical connector is configured and arranged to couple directly to an external trial stimulator.

It may be an advantage to use an electrical connector at the proximal end of the trial lead in lieu of employing terminals because fabricating terminals at the proximal end of the trial lead may be more labor intensive and expensive than coupling an electrical connector to the proximal end of the trial lead. It may also be an advantage to couple the electrical connector directly to the external trial stimulator because it eliminates the use of lead extensions (and, in some cases, operating room cables) during trial stimulations, thereby further reducing the cost, reducing the number of disposable items used during a trial stimulation, as well as reducing the number of potentially-unreliable connections. Eliminating the insertion and disposal of one or more lead extensions during a trial stimulation may also simplify the insertion procedure, and also reduce the environmental impact associated with the number of disposables used during the trial stimulation. Additionally, because the trial lead is long enough so that the electrical connector remains external to the patient during operation, then when, in at least some embodiments, one or more cables are used to couple the electrical connector to the external trial stimulator, the one or more cables may be reusable because the one or more cables remain external to the patient during operation.

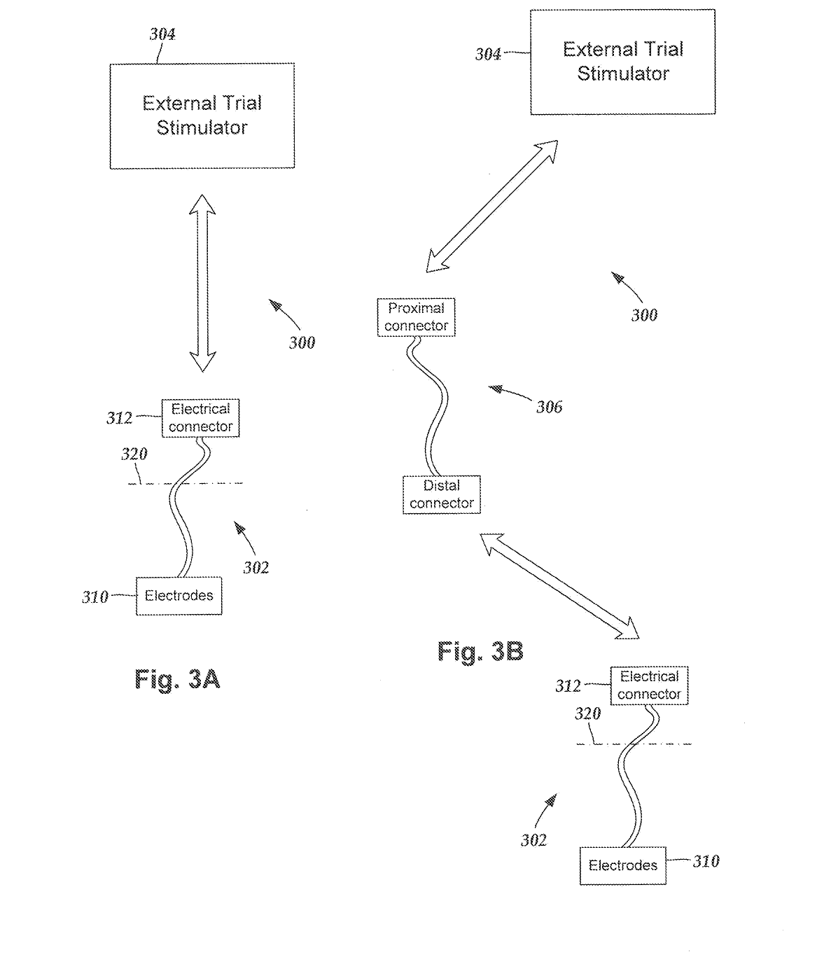

FIG. 3A is a schematic view of one embodiment of a trial system 300 that includes a trial lead 302 that is configured and arranged to couple directly to an external trial stimulator 304. FIG. 3B is a schematic view of another embodiment of the trial system 300 that includes the trial lead 302 and one or more cables 306 that couple to the trial lead 302 and that are configured and arranged to also couple to the external trial stimulator 304. The trial lead 302 includes electrodes 310 and an electrical connector 312. During operation, the electrodes 310 are disposed internal to the patient, while the electrical connector 312 remains external to the patient, as shown in FIGS. 3A and 3B by a line 320 schematically representing patient skin.

As shown in FIGS. 3A and 3B, the electrical connector 312 is configured and arranged to couple to the external trial stimulator 304. In at least some embodiments, the electrical connector 312 is configured and arranged to couple to the external trial stimulator 304 without using any lead extensions. In at least some embodiments, the electrical connector 312 is configured and arranged to couple directly to the external trial stimulator 304, as shown in FIG. 3A. In at least some embodiments, the electrical connector 312 is configured and arranged to couple to the external trial stimulator 304 via one or more cables 306, as shown in FIG. 3B.

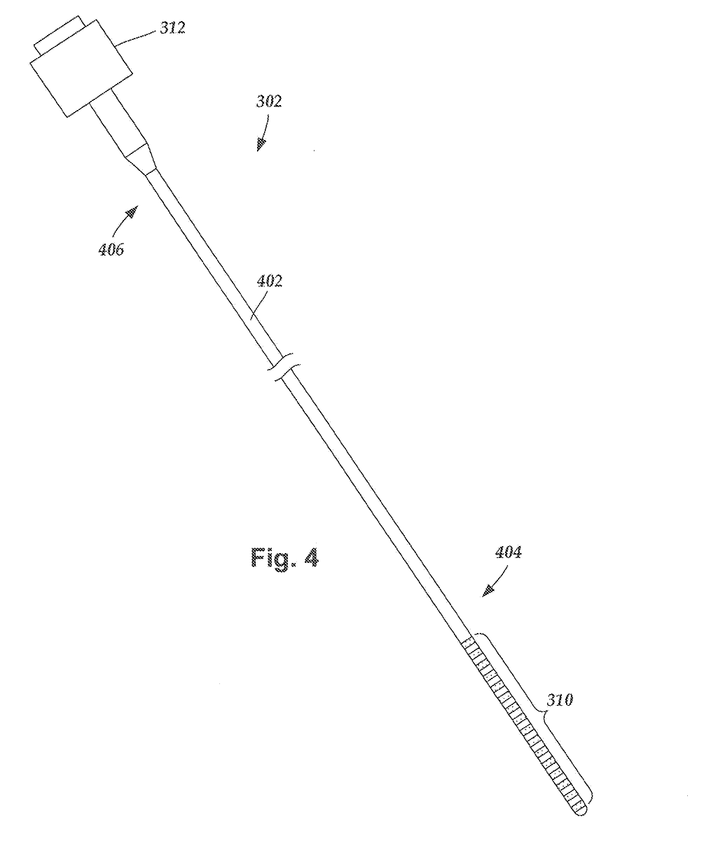

FIG. 4 is a schematic view of one embodiment of the trial lead 302. The trial lead 302 includes a lead body 402 having a longitudinal axis defined by a distal end 404 and a proximal end 406. In at least some embodiments, the plurality of electrodes 310 are disposed at the distal end 404 of the trial lead 302. In at least some embodiments, the electrical connector 312 is disposed at the proximal end 406 of the trial lead 302.

In at least some embodiments, the lead body 402 has a length of at least 70, cm, 80 cm, 90 cm, 100 cm, 110 cm, 120 cm, or more. In at least some embodiments, the lead body 402 has a length that is no longer than 140 cm, 130 cm, 120 cm, 110 cm, 100 cm, or less. In at least some embodiments, the lead body 402 has a length in the range of 80 cm to 140 cm. In at least some embodiments, the lead body 402 has a length in the range of 90 cm to 130 cm. In at least some embodiments, the lead body 402 has a length in the range of 100 cm to 120 cm.

In at least some embodiments, the lead body 402 is long enough to extend from a target stimulation region within a patient to a location external to the patient during operation. In at least some embodiments, the lead body 402 is long enough to extend from a target stimulation region within a patient to a connector of the one or more cables 306 that is positioned external to the patient and that is configured and arranged to couple the lead body 402 to the external trial stimulator 304. In at least some embodiments, the lead body 402 is long enough to extend from a target stimulation region within a patient to the external trial stimulator 304.

The trial lead 302 can include any number of electrodes 310 including, for example, two, three, four, five, six, seven, eight, nine, ten, eleven, twelve, fourteen, sixteen, twenty-four or more electrodes 310. It will be understood that other numbers of electrodes 310 may also be employed. The electrodes 310 are in electrical communication with the electrical connector 312 (e.g., via one or more conductors extending from the electrodes to the electrical connector 312).

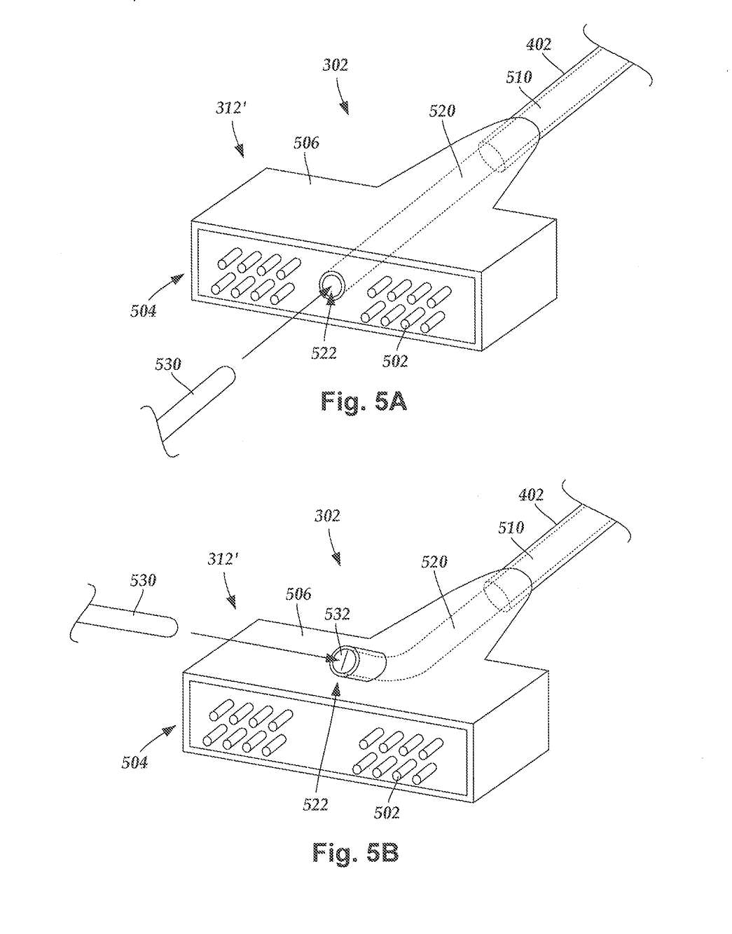

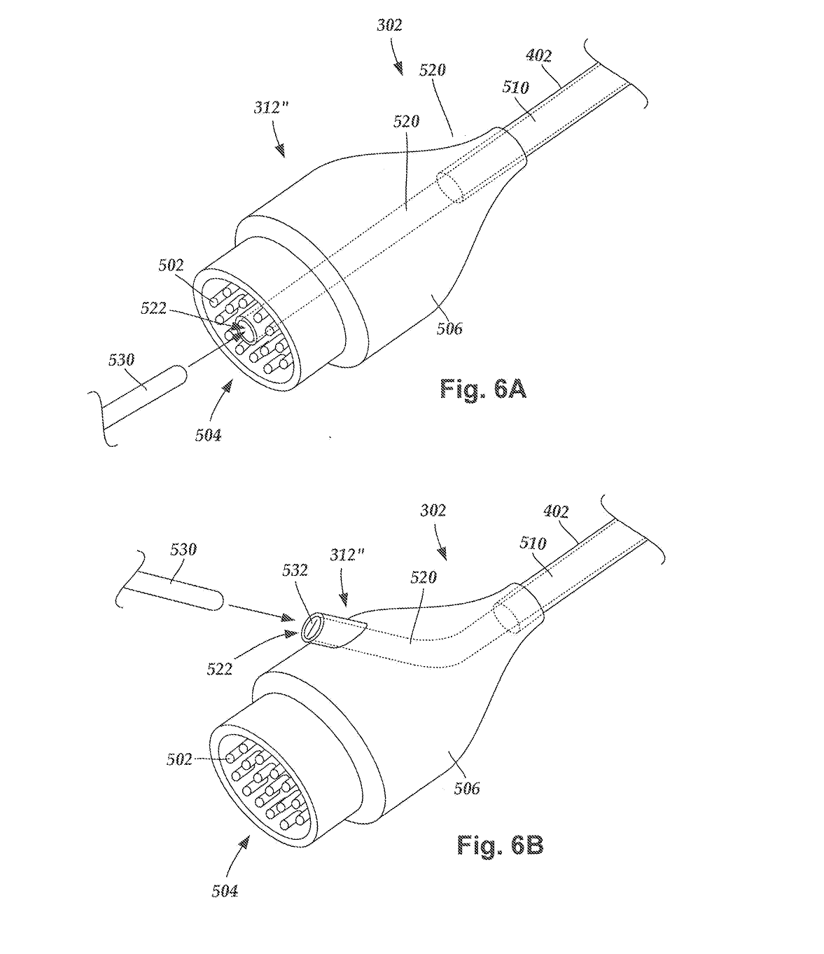

FIGS. 5A-6B illustrate several different exemplary embodiments of electrical connectors suitable for use with the trial lead 302. In at least some embodiments, the electrical connector includes one or more contacts, such as contact 502. A plurality of electrical conductors couple the electrodes 310 electrically to the contacts 502. In at least some embodiments, each of the electrodes 310 is coupled to at least one of the contacts 502. In at least some embodiments, the number of contacts is no fewer than the number of electrodes 310.

In at least some embodiments, the contacts 502 include one or more pins. In at least some embodiments, the contacts 502 include one or more pin receptacles. In at least some embodiments, the contacts 502 are arranged in a contact array. In at least some embodiments, the contacts 502 are arranged in a contact array that extends transversely to the longitudinal axis of the lead body 402. In at least some embodiments, a connector for commercially-available electronic devices is used as the electrical connector. For example, the electrical connector may be an HDMI connector, a LEMO connector, or the like.

FIGS. 5A-5B are schematic views of one embodiment of an electrical connector 312′ having contacts 502 configured into a rectangular array 504. FIGS. 6A-6B are schematic views of one embodiment of an electrical connector 312″ having contacts 502 configured into a round array. The contact array 504 is disposed at one end of a case 506 that couples to the proximal end 406 of the lead body 402. The contact array 504 can include any number of contacts 502 including, for example, two, three, four, five, six, seven, eight, nine, ten, eleven, twelve, fourteen, sixteen, twenty-four or more contacts 502. It will be understood that other numbers of contacts 502 may also be employed. FIGS. 5A-5B show the contact array 504 is formed as a rectangle. FIGS. 6A-6B show the contact array 504 is formed as a circle. It will be understood that the contact array 504 can be formed in any geometric or non-geometric shape suitable for coupling to a corresponding connector, such as a connector disposed on the one or more cables 306 or on the external trial stimulator 304.

In at least some embodiments, the lead body 402 defines a lumen 510 extending along at least a portion of a longitudinal axis of the lead body 402. In at least some embodiments, the lumen extends to the proximal end 406 of the lead body 402. In at least some embodiments, a connector lumen 520 couples to the lumen 510 at the proximal end 406 of the lead body 402 and extends outwardly therefrom to an access port 522.

In at least some embodiments, the trial system 300 includes a stylet 530 for guiding the electrodes 310 to a target stimulation region within the patient. In at least some embodiments, the stylet 530 is configured and arranged for insertion into the lumen 510 within the lead body 402. In at least some embodiments, the stylet 530 is configured and arranged for insertion into the lumen 510 via the connector lumen 520.

The access port 522 may be defined anywhere along an outer surface of the electrical connector 312. FIGS. 5A and 6A show the access port 522 defined along the contact array 504. FIGS. 5B and 6B show the access port 522 defined along a side surface of the case 506. In at least some embodiments, when the access port 522 is defined along a side surface of the case 506, an elastomeric self-sealing element, such as a split septum 532, is disposed over the access port 522 to prevent the flow of fluids into the lumen 510, as well as preventing ingress of contaminants into the access port 522, when the stylet 530 is not inserted into the connector lumen 520. In at least some embodiments, the septum is split to enable the stylet 530 access into the connector lumen 520, while maintaining a fluid-tight seal when the stylet 530 is not inserted into the connector lumen 520.

As discussed above, at least some trial stimulation leads are isodiametric to facilitate sliding of an epidural lead over a proximal end of the trial stimulation lead during removal of the epidural needle from a patient once the electrodes 310 are positioned. In at least some embodiments, the electrical connector 312 of the trial lead 302 has a circumference that is larger than a circumference of the lead body 402. Thus, the larger-sized electrical connector 312 may hinder, or even prevent, a conventional epidural needle from sliding off the proximal end of the trial lead 302.

In at least some embodiments, the trial system 300 further includes a lead introducer configured and arranged for facilitating insertion of a lead into a patient, including leads having non-isodiametric bodies, or leads having one or more larger-sized structures coupled thereto, such as at least some embodiments of the trial lead 302 and electrical connector 312. In at least some embodiments, the lead introducer of the trial system 300 includes a removable outer member configured and arranged to receive the trial lead during insertion of the trial lead into a patient.

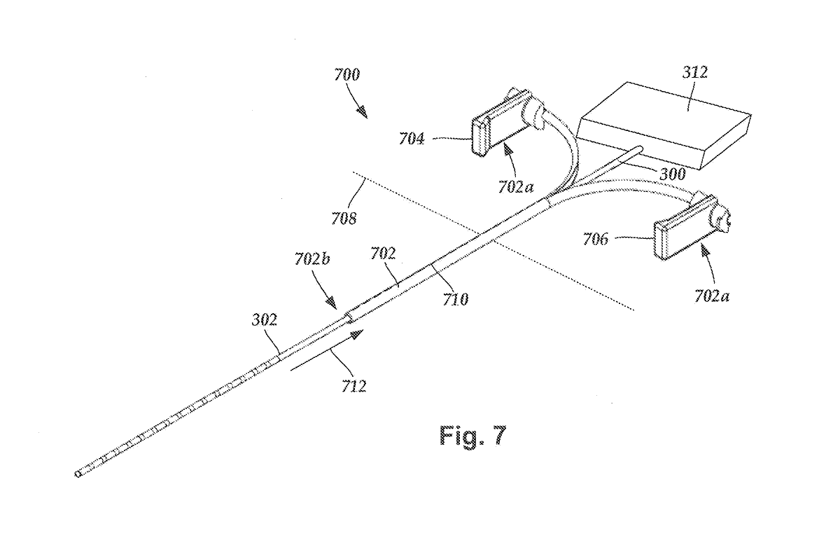

In at least some embodiments, the outer member is separatable from the trial lead by splitting apart. FIG. 7 is a schematic perspective view of one embodiment of a lead introducer 700 that includes a outer member 702 that splits to separate from the trial lead 302. The outer member 702 includes a proximal hub 702a having at least two pull-apart tabs 704 and 706.

In at least some embodiments, the outer member 702 is formed from a flexible material suitable for implantation into a patient 708 including, for example, fluorinated ethylene propylene, polytetrafluoroethylene, high-density polyethylene, polyetheretherketone, and the like or combinations thereof. Additionally, one or more radiopaque materials may be added including, for example, barium sulfate and bismuth subcarbonate, and the like or combinations thereof to facilitate implantation of the introducer sheath through the use of one or more medical imaging techniques, such as fluoroscopy.

In at least some embodiments, the outer member 702 includes one or more weakened regions 710, such as score lines or perforations, extending along at least a portion of a length of the outer member 702 from between the at least two pull-apart tabs 704 and 706. In at least some embodiments, when the at least two pull-apart tabs 704 and 706 are separated from one another, for example, by pulling each pull-apart tab away from the other pull-apart tab(s) in directions approximately orthogonal to the outer member 702, outer member 702 separates along the one or more weakened regions 710.

In at least some embodiments, outer member 702 is separated into a plurality of longitudinal strips while pulling the outer member 702 proximally along the trial lead 302. As the outer member 702 splits apart, the distal end 702b of the outer member 702 moves proximally along the trial lead 302 (as shown by arrow 712), with an increasing amount of the trial lead 302 extending through the distal end 702b of the outer member 702. In at least some embodiments, an undersurface of the outer member 702 includes a lubricious coating to facilitate the proximal movement of the outer member 702.

Eventually, the outer member 702 may be completely separated into two or more longitudinal strips, thereby separating completely from the trial lead 302 and also from the patient. In at least some embodiments, the distal ends of the outer member 702 may be extracted from the patient as the outer member 702 is split apart. In at least some embodiments, the outer member 702 may be split apart without causing the trial lead 302 to move.

In at least some embodiments, an insertion needle includes one or more body elements that receive the trial lead and that separate from one another after removal of the outer member. In at least some embodiments, separation of the one or more body elements enables removal of the body elements from the patient, while the trial lead 312 remains within the patient. In at least some embodiments, separation of the one or more body elements enables removal of the one or more body elements from the patient without sliding the insertion needle along the proximal end of the trial lead 302.

In at least some embodiments, the lead introducer includes an insertion needle configured and arranged to receive the trial lead and also configured and arranged for insertion into the outer member. In at least some embodiments, the insertion needle includes at least one body element that defines an open channel defined along a length of the insertion needle. In at least some embodiments, when the outer member is removed from the insertion needle, the trial lead laterally separates from the insertion needle by passing through the open channel.

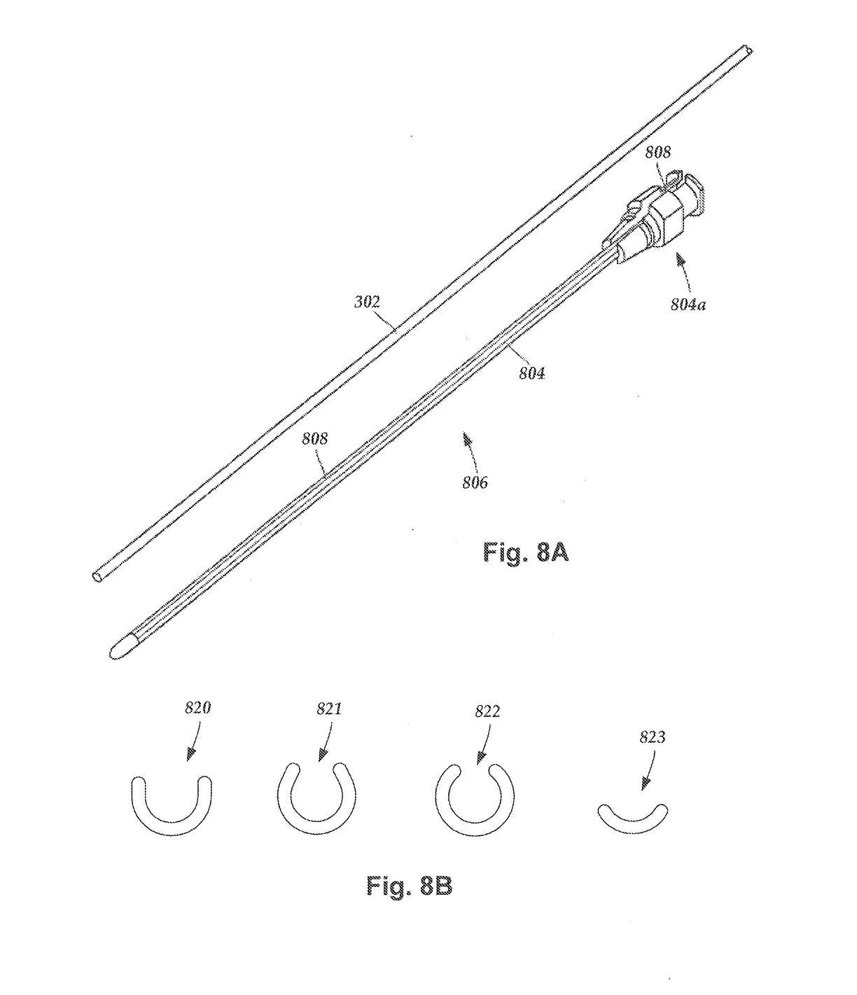

FIG. 8A is a schematic perspective view of one embodiment of the distal end of the trial lead 302 and a body element 804 of an insertion needle 806. The body element 804 defines an open channel 808 extending along a length of the body element 804. The open channel 808 is configured and arranged to receive the trial lead. In at least some embodiments, the open channel 808 extends substantially entirely along a length of the body element 804. In at least some embodiments, the open channel 808 extends along a proximal hub 804a of the body element 804. In at least some embodiments, the insertion needle 806 includes one more additional body elements.

In at least some embodiments, the open channel 808 is configured and arranged to receive the trial lead 302 during insertion of the trial lead 302 into the patient, and separate from the trial lead 302 during removal of the body element 804. In at least some embodiments, the open channel 808 separates from the trial lead 302 without moving the trial lead 302 axially relative to the body element 804 of the insertion needle 806. In at least some embodiments, the open channel 808 separates from the trial lead 302 by applying enough lateral force to at least one of the trial lead 302 or the body element 804 to pass the trial lead 302 out through the open channel 808. In at least some embodiments, the open channel 808 has a width that is no less than a diameter of the trial lead 302.

FIG. 8B is a schematic transverse cross-sectional view of several different exemplary embodiments of the open channel 808. In at least some embodiments, the portions of the body element 804 along which the open channel 808 extends have a transverse cross-sectional shape that is at least substantially U-shaped 820. In at least some embodiments, the portions of the body element 804 along which the open channel 808 extends have a transverse cross-sectional shape that is at least substantially horseshoe-shaped 821. In at least some embodiments, the portions of the body element 804 along which the open channel 808 extends have a transverse cross-sectional shape that is at least substantially C-shaped 822. In at least some embodiments, the portions of body element 804 along which the open channel 808 extends have a transverse cross-sectional shape that is at least substantially arc-shaped 823.

In at least some embodiments, the outer member 702 may be rolled or slid along a length of the trial lead or the insertion needle. In at least some embodiments, the lead introducer includes an insertion needle formed from a plurality of body elements and an outer member 702, such as heat shrink tubing, disposed over at least a portion of the insertion needle. In at least some embodiments, the insertion needle separates upon removal of the outer member. In some embodiments, the insertion needle may be separated from the trial lead when the body elements are partially separated from one another. In other embodiments, the insertion needle may be separated from the trial lead when the body elements are completely detached from one another.

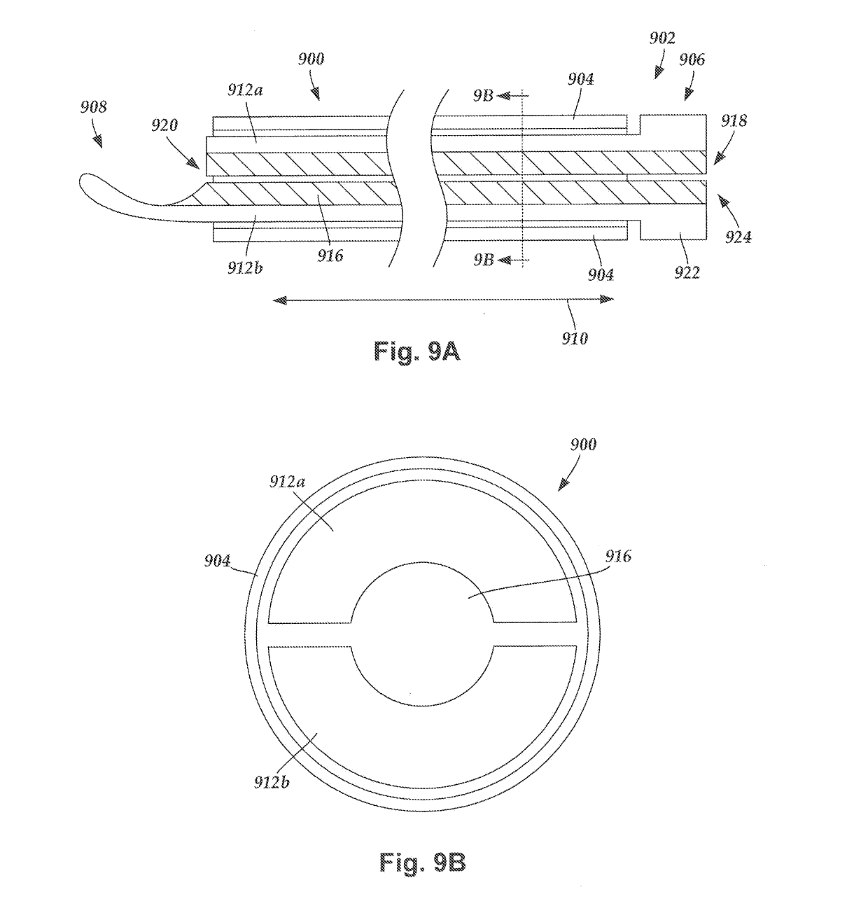

FIG. 9A is a schematic longitudinal cross-sectional view of one embodiment of a lead introducer 900 that includes an insertion needle 902 and an outer member 904 disposed over the insertion needle 302. FIG. 9B is a schematic transverse cross-sectional view of the lead introducer 900. The insertion needle 902 includes a proximal end 906, a distal end 908, and a longitudinal axis 910 (shown by a two-headed arrow). The insertion needle 902 also includes a plurality of body elements 912a and 912b mated together to define a lumen 916. In at least some embodiments, the body elements 912a and 912b are mated along the longitudinal axis 910 of the insertion needle 902. In at least some embodiments, the lumen 916 extends along the longitudinal axis 910. In at least some embodiments, the lumen 916 extends along the longitudinal axis 910 from the proximal end 906 to the distal end 908 of the insertion needle 902. In at least some embodiments, the lumen 916 extends from a proximal aperture 918 at the proximal end 906. In at least some embodiments, the lumen 916 extends from a distal aperture 920 at the distal end 908.

In at least some embodiments, the body elements are mated together within the outer member 904 such that the body elements 912a and 912b are at least partially separatable from one another when the outer member 904 is removed. In at least some embodiments, the body elements 912a and 912b at least partially separate from one another along a longitudinal axis of the insertion needle 902. In at least some embodiments, the body elements 912a and 912b separate from one another such that at least some of the plurality of body elements 912a and 912b remain coupled together. In at least some embodiments, the body elements 912a and 912b separate from one another such that at least some of the body elements 912a and 912b completely detach from one another. When the body elements 912a and 912b are separated (either partially or fully) from one another, the body elements 912a and 912b may be removed from the patient, leaving the trial lead 302 in place. In at least some embodiments, when the body elements 912a and 912b are separated (either partially or fully) from one another, the body elements 912a and 912b may be removed from the patient without sliding the insertion needle 902 off the proximal end of the trial lead 302 through the lumen of the lead introducer 900.

The outer member 904 may be formed from any thermoplastic material suitable for implantation including, for example, polyester, polyolefin, one or more fluoropolymers (such as fluorinated ethylene propylene, polytetrafluoroethylene, polyvinylidene fluoride, or the like or combinations thereof), polyvinyl chloride, polychloroprene, silicone elastomer, or the like or combinations thereof.

In at least some embodiments, the outer member 904 is disposed over at least a portion of an outer surface of the insertion needle 902. In at least some embodiments, the outer member 904 is disposed substantially entirely over the outer surface of the insertion needle 902 distal to the proximal hub 922. In at least some embodiments, the outer member 904 is disposed entirely over the outer surface of the insertion needle 902. In at least some embodiments, the outer member 904 forms a watertight seal along the lumen 916 of the insertion needle 902.

In at least some embodiments, once the outer member 904 is rolled or slid off the proximal end 906 of the insertion needle 902, the outer member 904 can be slid or rolled over the electrical connector 312. In at least some embodiments, the outer member 904 can be stretched to pass over the electrical connector 312. In at least some embodiments, the outer member 904 can be removed by cutting the outer member 904 along the longitudinal axis 910 of the outer member 904. In at least some embodiments, the outer member 904 can remain encircling the proximal end of the trial lead 302, external to the patient.

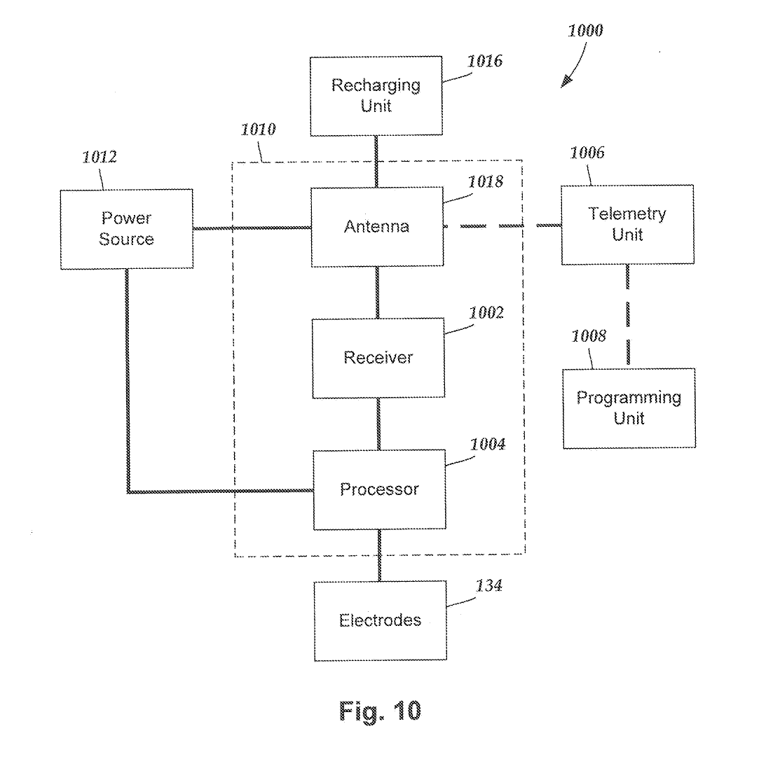

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A trial stimulation lead assembly for providing electrical stimulation of patient tissue during a trial stimulation, the trial stimulation lead assembly comprising:
a trial stimulation lead configured and arranged for insertion into a patient, the trial stimulation lead comprising
an elongated lead body having a length, a circumference, and a longitudinal axis defined by a proximal end and a distal end,
a plurality of electrodes disposed at the distal end of the lead body,
an electrical connector disposed at the proximal end of the lead body, the electrical connector comprising an outer case and a contact array disposed along the outer case, wherein the contact array extends transversely to the longitudinal axis of the lead body,
a plurality of electrical conductors extending along the length of the lead body and coupling each of the plurality of electrodes to at least one of the plurality of contacts, and
a lumen extending along at least a portion of the length of the lead body.

2. The trial stimulation lead assembly of claim 1, wherein the electrical connector has a circumference that is larger than the circumference of the lead body.

3. The trial stimulation lead assembly of claim 1, wherein the electrical connector further comprises a connector lumen defined in the electrical connector from a location adjacent the lumen of the lead body to an access port defined in the electrical connector, and wherein the lumen, connector lumen, and access port are configured and arranged for receiving a stylet.

4. The trial stimulation lead assembly of claim 3, wherein the access port is defined on the outer case of the electrical connector.

5. The trial stimulation lead assembly of claim 3, wherein the access port is defined within the contact array.

6. The trial stimulation lead assembly of claim 3, further comprising a self-sealing element disposed over the access port.

7. The trial stimulation lead assembly of claim 1, wherein the contact array is arranged into one of a rectangular configuration or a round configuration.

8. The trial stimulation lead assembly of claim 1, wherein the contact array comprises at least one of a plurality of pins or a plurality of pin receptacles.

9. The trial stimulation lead assembly of claim 1, wherein the contact array is one of a high definition multimedia interface connector or a LEMO connector.

10. The trial stimulation lead assembly of claim 1, wherein the number of contacts is no fewer than the number of electrodes.

11. The trial stimulation lead assembly of claim 1, wherein the contact array is configured and arranged to couple directly to an external trial stimulator.

12. The trial stimulation lead assembly of claim 1, wherein the contact array is configured and arranged to couple to an external trial stimulator via one or more operating room cables.

13. A kit for providing electrical stimulation of patient tissue during a trial stimulation, the kit comprising:
the trial stimulation lead assembly of claim 1; and
a lead introducer for facilitating insertion of the trial stimulation lead into the patient, the lead introducer comprising
an outer member configured and arranged for insertion into the patient, and
an insertion needle configured and arranged for insertion into the outer member, the insertion needle also configured and arranged to receive the distal end of the trial stimulation lead.

14. The kit of claim 13, further comprising a stylet for facilitating guidance of the electrodes to a target stimulation region within the patient, the stylet configured and arranged for insertion into the access port of the connector lumen of the trial stimulation lead assembly.

15. The kit of claim 13, wherein the outer member is configured and arranged to divide into at least two parts for removal of the outer member from the trial stimulation lead upon insertion of the trial stimulation lead into the patient.

16. The kit of claim 13, wherein the insertion needle defines an open channel configured and arranged to receive at least a portion of the trial stimulation lead.

17. The kit of claim 13, wherein the insertion needle comprises a plurality of body elements configured and arranged to at least partially separate from one another upon removal of the outer member.

18. A method for implanting a trial electrical stimulation into a patient, the method comprising:
inserting an insertion needle into an outer member;
guiding a distal end of the outer member to a target stimulation region within the patient;
inserting the distal end of the trial stimulation lead of claim 1 into the insertion needle;
removing the outer member from the patient while leaving the trial stimulation lead within the patient such that the plurality of electrodes are at the target stimulation region;
separating the trial stimulation lead from the insertion needle; and
removing the insertion needle from the trial stimulation lead while leaving the trial stimulation lead within the patient such that the plurality of electrodes are at the target stimulation region.

19. The method of claim 18, wherein removing the outer member from the patient comprises separating the outer member into at least two parts along a length of a lumen of the outer member and removing the outer member from the patient.

20. The method of claim 18, wherein removing the insertion needle from the trial stimulation lead comprises passing the trial stimulation lead laterally through an open channel of the insertion needle and removing the insertion needle from the patient.

21. The method of claim 18, wherein removing the insertion needle from the trial stimulation lead comprises at least partially separating at least two of a plurality of body elements from one another along a longitudinal axis of the insertion needle.

22. The method of claim 18, wherein removing the outer member from the patient comprises at least one of rolling up or sliding the outer member proximally along the longitudinal axis of the insertion needle.
